(19) United States
(12) Patent Application Publication
(10) Pub. No.: US 2008/0281221 A1
(43) Pub. Date: Nov. 13, 2008

(54) Method and Apparatus for Encouraging Physiological Change Through Physiological Control of Video Devices

(76) Inventors: Devon Greco, San Marcos, CA (US); Domenic Greco, San Marcos, CA (US)

Correspondence Address: WHITE-WELKER & WELKER, LLC, P.O. BOX 199, CLEAR SPRING, MD, 21722-0199, US

(21) Appl. No.: 12/118,701

(22) Filed: May 10, 2008

Related U.S. Application Data
(60) Provisional application No. 60/917,159, filed on May 10, 2007.

(51) Int. Cl. A61B 5/0482

(52) U.S. Cl. 600/545

(57) ABSTRACT

The present invention concerns a method and system for encouraging physiologic change through physiological display control of video devices. The system includes interfacing an output from a physiologic acquisition device with a junction box that is capable of controlling the output of the physiologic acquisition device which can be an EEG, and whose output can be analog or digital, visually modulated output of the EEG signal or can be an audio feedback output of the EEG training signal. In one embodiment of the present invention, the PC is no longer required to process the audio/visual changes affected on the video device. The junction box can include means for changing the video feed characteristics such as brightness or darkness of the video image and also volume control of the video feed in response to changes in the signals of brain activity.

### CROSS REFERENCE TO RELATED APPLICATIONS

This application claims priority from U.S. Provisional Patent Application Ser. No. 60/917,159, entitled “Method and Apparatus for Encouraging Physiological Change Through Physiological Control of Video Devices”, filed on 10 May 2007.

### FEDERALLY SPONSORED RESEARCH

Not Applicable

### SEQUENCE LISTING OR PROGRAM

Not Applicable

### TECHNICAL FIELD OF THE INVENTION

The present invention relates generally to the use of physiological means in video devices. More specifically, the present invention relates to display control of video devices using physiological means for encouraging physiological change.

### BACKGROUND OF THE INVENTION

The term ‘Biofeedback’ refers to techniques in which an individual learns to consciously control involuntary responses such as heart rate, brain waves, anxiety and muscle tension, with the help of man-machine interfaces such as computer screens. In the case of humans, biofeedback is artificially mediated by man-made detection, amplification, and display instrumentation as we lack the presence of an inborn feedback loop within the biological system.

Biofeedback systems relay information about normally unconscious physiologic processes back to the individual in the form of visual, auditory, or tactile signals. These responses can be electronically monitored and noted through beeps, graphs, or other visual representations, including displays on computer screens.

Biofeedback systems can be used for a number of applications, one of them being towards addressing chronic, persistent developmental and behavioral disorders like Attention Deficit Hyperactivity Disorder (ADHD), Learning Disabilities, Cognitive Effects of Aging and other cognitive disorders. People with these disorders have severe difficulty in focusing and maintaining attention. Characteristically, those suffering from these disorders display inattentiveness, impulsiveness, and hyperactivity. These disorders often lead to learning and behavior problems at home, school or work for those people. Generally, biofeedback systems can be used towards addressing any cognitive processing disorder, learning disability, anxiety, depression, mild closed head injury and cognitive effects of aging and the like as these can respond favorably to treatment using biofeedback and specifically EEG biofeedback.

Such disorders are currently treated using a variety of methods, including the use of medication, behavioral therapy, audio-visual entrainment, cerebella function stimulation and brainwave biofeedback training to manage the symptoms. Biofeedback and EEG Biofeedback training use machines to measure and display body functions and states such as heart rate, skin temperature, muscle tension, brain activity, Electroencephalograph (EEG), Electromyograph (EMG) and skin conductance. The patient can monitor these body functions and see how and why the body functions change through stages of high and low degrees of activity, with the goal that the patient eventually learns to control those body functions.

Biofeedback and EEG Biofeedback training allows the patient to monitor and improve his/her level of attention or symptoms by observing the machine that measures and displays their body functions, making the patient aware of the activities which promote improvement, thus reinforcing the patient's ability to control the body functions.

Also, in today's technologically advanced work environments, it is often the ability and performance of the human component that acts as the limiting factor in the proper performance of tasks, being as it is, susceptible to stress, decreased cognitive efficiency and lack of attentiveness and aging. In such technologically advanced and demanding work environments, the use of biofeedback has typically been a cumbersome task, as it requires the subject to pay too much attention to the biofeedback signals being presented to him, leaving little scope for him to be able to accomplish the requisite work-tasks. It is necessary for the worker to be able to control physiological activity such as stress, lack of awareness, fluctuations of his degree of attention etc, as work environments that require higher degrees of awareness would require him to be able to maintain an efficient physiological state, and be capable of quickly recovering from any lapses of attention or other stresses that may occur.

Technologies known in the art describe methods for improving attention skill by rewarding specific brain signal patterns with desirable results such as success at playing a video game or altering the characteristics of the display of a video feed in a desirable manner. The player or viewer is required to exhibit the required brain signal patterns that accompany normal behavior in order to win the video game or alter a simple computer animation desirably, as opposed to exhibiting behavior consistent with someone suffering from ADD or ADHD. Once the player or viewer exhibits the required brain signal patterns, the video game or computer generated animation becomes easier to play or advances. A measurement system senses the EEG signals from the player and routes them to the computer where the video game difficulty is computed or the video feed characteristics are determined, therefore varying the difficulty of the video game or view-ability of the video feed.

The known technologies that use video feeds such as DVD movies have the disadvantage of requiring extensive equipment, typically requiring a Personal Computer (PC) that is interfaced with video playback systems such as PC media players or external DVD players and feed the brain activity mediated signal to the PC screen or a Television display. This requirement of a PC causes significant compatibility problems in an end user setup, often due to the varied versions and types of audio and video Coders/Decoders (CODECS) present on such systems. A PC mediated system also complicates the use of the training system for an end user especially in the elderly.

### SUMMARY OF THE INVENTION

The present invention concerns a method and system for encouraging physiologic change through physiological display control of video devices. The system includes interfacing an output from a physiologic acquisition device with a junction box that is capable of controlling the output of the physiologic acquisition device which can be an EEG, and whose output can be analog or digital, visually modulated output of the EEG signal or can be an audio feedback output of the EEG training signal. In one embodiment of the present invention, the PC is no longer required to process the audio/visual changes affected on the video device.

In one embodiment of the present invention, multiple signal outputs acquired from the physiologic acquisition device are sent to the junction box. The acquired signals can be sent utilizing at least one of a wireless or a wired interface. The junction box interfaces with a video playback device such as a DVD player and/or a Television set. The output signals from the physiologic acquisition device can modulate multiple signals of brain activity, such as Sensory Motor Rhythm (SMR), Delta waves, Theta waves, Alpha waves, Beta waves, and Gamma waves that are sent to the junction box.

The junction box can include means for changing the video feed characteristics such as brightness or darkness of the video image and also volume control of the video feed in response to changes in the signals of brain activity. In an embodiment of the present invention, the means for changing the video feed characteristics can be potentiometers. The changes in the video feed characteristics encourage the viewer to produce brain activity that represents improved brain function through reinforcing and/or inhibiting certain brain wave activity.

It is also an object of the present invention to teach another embodiment of the present invention off-the-shelf standalone video devices such as portable DVD players can be utilized to modulate the video feed characteristics. In another embodiment of the invention, a standalone DVD player can be used in conjunction with a TV. In still another embodiment of the invention, the signal that is being sent to a TV can be directly modulated. In an embodiment of the present invention, a microchip can be utilized to modulate multiple voltage outputs. These multiple voltage outputs can act as a variable resistor or potentiometer, and can be integrated into the video display unit of a portable DVD player. This can be utilized as variable impedance that can be used in place of the potentiometers or other means already in place on the portable DVD player that are used for volume control, brightness or contrast control and the like.

It is also an object of the present invention to teach an embodiment of the present invention wherein the junction box can be configured to interface between a plurality of devices such as a DVD player, VHS Player, a Cable Box and a Television. The junction box can be powered by an external power source. Alternatively, the junction box can support at least one of a wired or wireless output interface. In an exemplary configuration, a viewer can use an NTSC signal if based in North America or can use PAL signal if based in Europe and Asia. The junction box can connect to the other components using standard interfaces such as RCA jacks and S-Video.

The interface from the junction box would be used to modulate the brightness/contrast control of the device video signal while another output would be used to modulate the volume control of the signal based upon real time physiologic measurements.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is a diagram that shows an exemplary prior art configuration; and

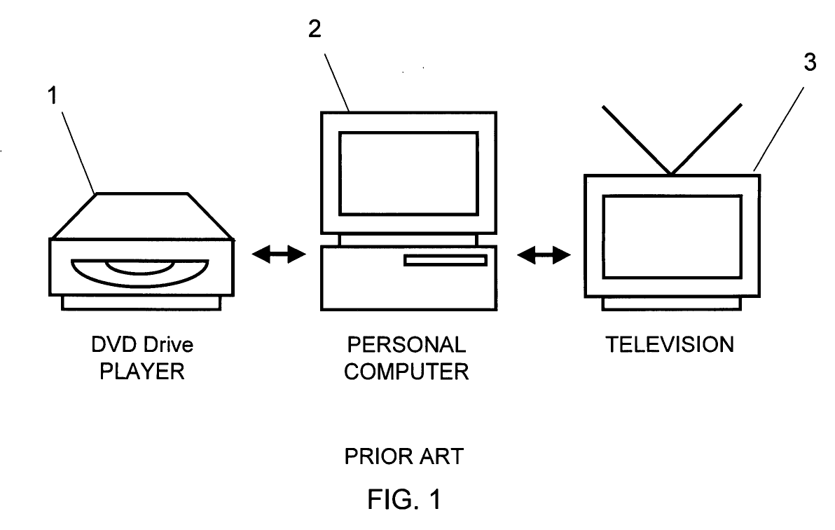

FIG. 2 is a diagram that shows an exemplary system configuration, illustrated in accordance with some embodiments of the present invention.

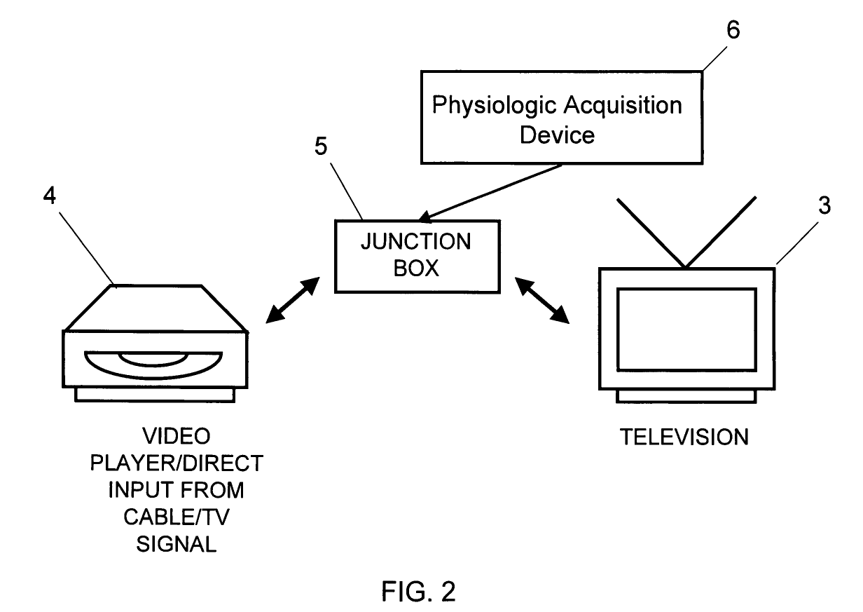

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention.

Referring to the figures, it is possible to see the various major elements constituting the apparatus of the present invention. Now referring to Figures, the embodiment of the Method and Apparatus for Encouraging Physiological Change Through Physiological Control of Video Devices is illustrated.

The present invention concerns a method and system for encouraging physiologic change through physiological display control of video devices. The system includes interfacing an output from a physiologic acquisition device 6 with a junction box 5 that is capable of controlling the output of the physiologic acquisition device 6 which can be an EEG, and whose output can be analog or digital, visually modulated output of the EEG signal or can be an audio feedback output of the EEG training signal. In one embodiment of the present invention, the personal computer 2 is no longer required to process the audio/visual changes affected on the video device 4.

In another embodiment of the present invention, multiple signal outputs acquired from the physiologic acquisition device 6 are sent to the junction box 5. The acquired signals can be sent utilizing at least one of a wireless or a wired interface. The junction box 5 interfaces with a video playback device such as a DVD player 1 and/or a Television set 3. The output signals from the physiologic acquisition device 6 can modulate multiple signals of brain activity, such as Sensory Motor Rhythm (SMR), Delta waves, Theta waves, Alpha waves, Beta waves, and Gamma waves that are sent to the junction box 5.

The junction box 5 can include means for changing the video feed characteristics such as brightness or darkness of the video image and also volume control of the video feed in response to changes in the signals of brain activity. In an embodiment of the present invention, the means for changing the video feed characteristics can be potentiometers. The changes in the video feed characteristics encourage the viewer to produce brain activity that represents improved brain function through reinforcing and/or inhibiting certain brain wave activity.

In yet another embodiment of the present invention off-the-shelf standalone video devices such as portable DVD players 4 can be utilized to modulate the video feed characteristics. In another embodiment of the invention, a standalone DVD player 4 can be used in conjunction with a TV 3. In still another embodiment of the invention, the signal that is being sent to a TV 3 can be directly modulated.

In another embodiment of the present invention, a microchip can be utilized to modulate multiple voltage outputs. These multiple voltage outputs can act as a variable resistor or potentiometer, and can be integrated into the video display unit of a portable DVD player 1. This can be utilized as variable impedance that can be used in place of the potentiometers or other means already in place on the portable DVD player 1 that are used for volume control, brightness or contrast control and the like.

In another embodiment of the present invention, the microchip can be placed in a standalone project box and can derive power from an internal battery of the portable DVD player 1.

In an embodiment of the present invention, the junction box 5 can be configured to interface between a plurality of devices such as a DVD player, VHS Player, a Cable Box 4 and a Television 3. The junction box 5 can be powered by an external power source.

In an embodiment of the present invention, the junction box 5 can support at least one of a wired or wireless output interface. In an exemplary configuration, a viewer can use an NTSC signal if based in North America or can use PAL signal if based in Europe and Asia. The junction box 5 can connect to the other components using standard interfaces such as RCA jacks and S-Video.

The interface from the junction box 5 would be used to modulate the brightness/contrast control of the device video signal while another output would be used to modulate the volume control of the signal based upon real time physiologic measurements.

Thus, it is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. An apparatus for encouraging physiologic change through physiological display control of video devices consisting of the following parts in combination:
a physiologic acquisition device;
a junction box;
means for interfacing an output from a physiologic acquisition device with the junction box; and
wherein said junction box is capable of controlling the output of the physiologic acquisition device.

2. The apparatus of claim 1 wherein the physiologic acquisition device is an EEG whose output can be
analog or digital;
visually modulated output of the EEG signal; or
an audio feedback output of the EEG training signal.

3. The apparatus of claim 1 wherein
multiple signal outputs acquired from the physiologic acquisition device are sent to the junction box;
the acquired signals are sent utilizing one or more interfaces;
the junction box interfaces with a playback device;
the output signals from the physiologic acquisition device can modulate multiple signals of brain activity.

4. The apparatus of claim 3 wherein the interface may be wireless or wired.

5. The apparatus of claim 3 wherein the playback device is a DVD player, Television set, computer, or other personal electronic device.

6. The apparatus of claim 3 wherein the playback device supports either audio, video, or a combined audio and visual information.

7. The apparatus of claim 3 wherein the output signals from the physiologic acquisition device can modulate multiple signals of brain activity, including
Sensory Motor Rhythm (SMR);
Delta waves;
Theta waves;
Alpha waves;
Beta waves; and
Gamma waves
that are sent to the junction box.

8. The apparatus of claim 3 wherein the junction box can includes means for
changing the video feed characteristics of a video image, and
volume control of a video feed
in response to changes in the signals of brain activity.

9. The apparatus of claim 3 wherein the means for changing the video feed characteristics can be potentiometers.

10. The apparatus of claim 3 wherein standalone video devices are utilized to modulate the video feed characteristics.

11. The apparatus of claim 10 wherein, a standalone DVD player is used in conjunction with a TV.

12. The apparatus of claim 10 wherein, the signal that is being sent to a TV can be directly modulated by a microchip utilized to modulate multiple voltage outputs; and said multiple voltage outputs act as a variable resistor or potentiometer, in a standalone video device.

13. The apparatus of claim 12 wherein the microchip is placed in a standalone project box and can derive power from an internal battery of a standalone video device.

14. The apparatus of claim 1 wherein the junction box is configured to interface between a plurality of standalone video device devices.

15. The apparatus of claim 1 wherein the interface from the junction box modulates the brightness and contrast control of the device's video signal while another output would be used to modulate the device's volume control of the signal based upon real time physiologic measurements.

16. A method for encouraging physiological change through physiological control of video devices comprising the steps of
providing one or more physiologic acquisition devices;
providing a junction box;
interfacing an output from one or more physiologic acquisition devices with the junction box; and
controlling the output of one or more physiologic acquisition devices by the junction box.

17. The method of claim 16 further comprising the steps of visually modulating the output of the EEG signal and an audio feedback output of the EEG training signal.

18. The method of claim 17 further comprising the steps of
acquiring from the physiologic acquisition device multiple signal outputs;
utilizing one or more interfaces to send said multiple signal outputs to the junction box;
sending said multiple signal outputs to the junction box;
interfacing the junction box with a playback device; and
modulating multiple output signals of brain activity from the physiologic acquisition device.

19. The method of claim 18 further comprising the steps
changing the video feed characteristics of a video image, and volume control of a video feed in response to changes in the signals of brain activity; and
providing potentiometers as the means for changing the video feed characteristics

20. The method of claim 19 further comprising the steps modulates the brightness and contrast control of the device's video signal while another output would be used to modulate the device's volume control of the signal based upon real time physiologic measurements.
